(19) United States
(12) Patent Application Publication
(10) Pub. No.: US 2009/0053582 A1
(43) Pub. Date: Feb. 26, 2009

(54) METHOD OF COATING BILIRUBIN OXIDASE

(75) Inventors: Taiki Sugiyama, Kanagawa (JP); Yoshio Goto, Kanagawa (JP); Yuichi Tokita, Kanagawa (JP)

Correspondence Address: BELL, BOYD & LLOYD, LLP, P. O. BOX 1135, CHICAGO, IL, 60690, US

(73) Assignee: SONY CORPORATION, Tokyo (JP)

(21) Appl. No.: 12/192,715

(22) Filed: Aug. 15, 2008

(30) Foreign Application Priority Data
Aug. 20, 2007 (JP) 2007-213512

(51) Int. Cl. H01M 8/16; C12N 9/96; C12N 9/02

(52) U.S. Cl. 429/43; 435/188; 435/189

(57) ABSTRACT

A method of coating bilirubin oxidase (BOD) is provided and includes introducing a polymerizable functional group onto the surface of a bilirubin oxidase (BOD) molecule, and copolymerizing the polymerizable functional group with a polymerizable monomer. The step of introducing the polymerizable functional group and the step of copolymerizing the polymerizable functional group with the polymerizable monomer are performed at 17° C. or lower.

### CROSS REFERENCES TO RELATED APPLICATIONS

The present application claims priority to Japanese Patent Application JIP 2007-213512 filed in the Japanese Patent Office on Aug. 20, 2007, the entire contents of which is incorporated herein by reference.

### BACKGROUND

The present application relates to a method of coating bilirubin oxidase (BOD). More specifically, the present application relates to a method of coating bilirubin oxidase while suppressing a decrease in the enzyme activity, a method of stabilizing bilirubin oxidase utilizing the coating method, bilirubin oxidase coated using the coating method, an enzyme-immobilized electrode on which the coated bilirubin oxidase is immobilized, and a fuel cell including the enzyme-immobilized electrode.

Bilirubin oxidase (BOD) is an enzyme that belongs to a group of multicopper oxidase (which is the generic name of an enzyme having a plurality of copper ions as an active center), and that catalyzes an oxidation reaction in which bilirubin becomes biliverdin. This enzyme has been widely used as a test reagent of, for example, the liver function (a reagent for measuring bilirubin in serum) in clinical tests. For example, Japanese Unexamined Patent Application Publication No. 2004-194670 discloses a method of measuring the concentration of direct bilirubin by determining an optical change in a sample when the sample is treated with bilirubin oxidase in the presence of 100 to 800 mM of potassium ions.

Enzymes such as bilirubin oxidase are biocatalysts that generally help facilitate many reactions related to the maintenance of life under mild conditions in a living body. Recently, techniques for utilizing such enzymes in vitro have attracted attention. For example, techniques related to use of enzymes in various technical fields such as production of useful materials, production, measurement, and analysis of energy-related substances, environmental protection, and medical treatment, and techniques related to biofuel cell, which is one example of fuel cells, an enzyme-immobilized electrode, and an enzyme sensor (a sensor for measuring the concentration of a chemical substance by utilizing an enzymatic reaction) have also been developed.

Bilirubin oxidase has also attracted attention as a catalyst that realizes an electrochemical four-electron reduction reaction of oxygen at the cathode (positive electrode) side of biofuel cell. For example, Japanese Unexamined Patent Application Publication No. 2007-87627 discloses a fuel cell in which bilirubin oxidase is immobilized on a positive electrode.

Enzymes such as bilirubin oxidase are proteins, and thus have a property that they are easily denatured by an effect of, for example, heat, pH, and organic solvents. Therefore, the stability of enzymes in vitro is lower than that of other chemical catalysts such as a metal catalyst. Accordingly, in the case where enzymes are used in vitro, it is important that the stability be increased while maintaining the activity.

The following techniques are disclosed as methods of stabilizing bilirubin oxidase. For example, Japanese Unexamined Patent Application Publication No. 2000-83661 discloses a method of stabilizing bilirubin oxidase (BOD) in which a pentacyanoiron complex and/or a hexacyanoiron complex, and a tertiary amine are added to a BOD-containing solution. Japanese Unexamined Patent Application Publication No. 2000-253873 discloses a method of stabilizing bilirubin oxidase in which a lithium compound or homocystine is used as a stabilizer. Japanese Unexamined Patent Application Publication No. 2004-89042 discloses heat-resistant bilirubin oxidase produced by a genetic engineering method.

A coating technique related to an embodiment of the present application will be now described.

Recently, in a wide range of fields such as the field of medicine, the field of cosmetics, and the field of food production, techniques for coating a physiologically active substance, such as a protein, with a polymer or the like have been developed. A typical example thereof is a drug delivery system. These techniques are used for various purposes such as control of an active site, transportation in a living body, improvement in the stability and retainment of quality, and control of reactivity. Specifically, according to such techniques, for example, a physiologically active substance is coated with a polymer or the like so that the physiologically active substance is released at a desired active site or the like to cause an active reaction.

As a novel technique following on from these techniques, J. Am. Chem. Soc. 2006, 128, 11008-11009 (Document 1) discloses a technique for allowing horseradish peroxidase (HRP) molecules to be individually enclosed in a nano-gel, in which thermostability and organic solvent resistance can be improved while maintaining the enzyme activity. This technique has a feature that the enzyme molecules are individually enclosed in a nano-gel, and the enzyme activity is not decreased after the enclosure. However, whether or not this technique can be applied to physiologically active substances other than horseradish peroxidase (HRP) is unknown, and further room for study still remains.

### SUMMARY

It is desirable to provide a novel method of coating bilirubin oxidase (BOD) while suppressing a decrease in the enzyme activity of bilirubin oxidase.

Attempts to coat bilirubin oxidase using the technique disclosed in Document 1 resulted in that bilirubin oxidase could be successfully coated, but the enzyme activity was significantly decreased. The present embodiments provide a method of coating bilirubin oxidase while suppressing a decrease in the enzyme activity.

According to an embodiment, there is provided a method of coating bilirubin oxidase (BOD) including the steps of introducing a polymerizable functional group onto the surface of a bilirubin oxidase molecule, and copolymerizing the polymerizable functional group with a polymerizable monomer, wherein the step of introducing the polymerizable functional group and the step of copolymerizing the polymerizable functional group with the polymerizable monomer are performed at 17° C. or lower.

Hitherto, according to a general technical concept, each of the above steps, in particular, the step of copolymerizing the polymerizable functional group with the polymerizable monomer has been conducted only at high temperatures higher than a certain temperature. In contrast, in an embodiment, this idea is significantly changed, and these steps can be successfully performed at relatively low temperatures of 17° C. or lower.

Each of the steps are performed at 17° C. or lower, and more preferably, at 4° C. or higher but 17° C. or lower.

The polymerizable functional group is not particularly limited as long as the polymerizable functional group can be introduced onto the surface of a bilirubin oxidase molecule. An example of the polymerizable functional group is an acryloyl group.

The polymerizable monomer is not particularly limited as long as the polymerizable monomer can be polymerized with the polymerizable functional group. An example of the polymerizable monomer is acrylamide.

By employing the coating method according to an embodiment, bilirubin oxidase can be stabilized.

For example, by improving thermostability, bilirubin oxidase can be stabilized.

Alternatively, by improving the resistance against an organic solvent, bilirubin oxidase (BOD) can be stabilized. The organic solvent is not particularly limited. For example, by improving methanol resistance, bilirubin oxidase can be stabilized.

Also, according to an embodiment, there is provided a bilirubin oxidase coated by the method according to an embodiment.

The coated bilirubin oxidase (BOD) can be used for any applications. For example, the coated bilirubin oxidase (BOD) can be immobilized on an enzyme-immobilized electrode and used as a catalyst.

According to an embodiment, there is further provided a fuel cell including at least the enzyme-immobilized electrode.

By employing the coating method according to an embodiment, bilirubin oxidase molecules can be individually coated while suppressing a decrease in the enzyme activity.

Additional features and advantages are described herein, and will be apparent from the following Detailed Description and the figures.

### BRIEF DESCRIPTION OF THE FIGURES

FIG. 1 is a schematic view showing a method of coating bilirubin oxidase (BOD) according to an embodiment;

FIG. 2 includes graphs showing the relationship between the substrate concentration and the enzyme activity of coated BOD 1, coated BOD 2, and free BOD 1, the relationship being obtained by performing fitting using the Michaelis-Menten equation;

FIG. 3 includes graphs showing the relationship between the heat treatment time and the enzyme activity of coated BOD 1, coated BOD 2, and free BOD 1, the relationship being obtained by performing fitting using the rate analysis equation represented by equation 1;

FIG. 4 is a graph showing the relationship between the heat treatment time and the residual activity of coated BOD 1, coated BOD 2, and free BOD 1, the relationship being obtained by performing fitting using the rate analysis equation represented by equation 1;

FIG. 5 is a graph showing the relationship between the substrate concentration and the enzyme activity of coated BOD 3, coated BOD 4, free BOD 2, and free BOD 3, the relationship being obtained by performing fitting using the Michaelis-Menten equation;

FIG. 6 is a graph showing the relationship between the heat treatment time and the enzyme activity of coated BOD 3, coated BOD 4, free BOD 2, and free BOD 3, the relationship being obtained by performing fitting using the rate analysis equation represented by equation 1;

FIG. 7 is a graph showing the relationship between the heat treatment time and the residual activity of coated BOD 3, coated BOD 4, free BOD 2, and free BOD 3, the relationship being obtained by performing fitting using the rate analysis equation represented by equation 1;

FIG. 8 is a graph showing the relationship between the heat treatment temperature and the enzyme activity of coated BOD 3, coated BOD 4, free BOD 2, and free BOD 3;

FIG. 9 is a graph showing the relationship between the heat treatment temperature and the residual activity of coated BOD 3, coated BOD 4, free BOD 2, and free BOD 3;

FIG. 10 is an enlarged graph showing the range from 50° C. to 70° C. in FIG. 9;

FIG. 11 is a graph showing the relationship between the temperature T (° C.) during a process of coating BOD and the ratio of residual activity of BOD; and

FIG. 12 is a graph showing the residual activity of each BOD sample after an organic solvent treatment.

### DETAILED DESCRIPTION

Preferred embodiments will now be described with reference to the drawings. It should be understood that the embodiments described below show examples of typical embodiments, and the scope of the present application is not limited by the embodiments.

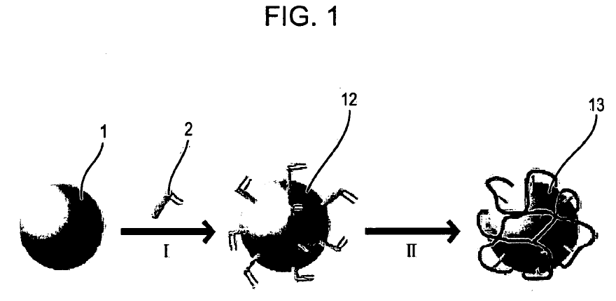

FIG. 1 is a schematic view showing a method of coating bilirubin oxidase (BOD) according to an embodiment. Symbol I denotes a step of introducing a polymerizable functional group in the coating method according to the embodiment. Symbol II denotes a step of copolymerization. Reference numeral 1 indicates a bilirubin oxidase (BOD) molecule, reference numeral 2 indicates a polymerizable functional group, reference numeral 12 indicates a bilirubin oxidase (BOD) molecule which has had polymerizable functional groups introduced onto the surface of the molecule, and reference numeral 13 indicates a coated bilirubin oxidase (BOD) molecule.

Step I of Introducing Polymerizable Functional Group

In the coating method according to this embodiment, first, a step of introducing the polymerizable functional group 2 onto the surface of a bilirubin oxidase (hereinafter referred to as “BOD”) molecule is performed. Any method in the related art can be used as the method of introducing the polymerizable functional group 2. For example, the polymerizable functional group 2 can be introduced onto the surface of the BOD molecule by covalently bonding an organic compound having the polymerizable functional group 2 to an amino group present on the surface of the BOD molecule.

Step I of introducing a polymerizable functional group is performed at 17° C. or lower in order to suppress a decrease in the enzyme activity of BOD. Hitherto, introduction of such a polymerizable functional group 2 has been generally performed at about 30° C. However, in the embodiment, the idea of the related art is significantly changed, and by introducing the polymerizable functional group 2 at a relatively low temperature of 17° C. or lower, the decrease in the enzyme activity of BOD can be successfully suppressed.

When step I of introducing a polymerizable functional group is performed at 17° C. or lower, the decrease in the enzyme activity of BOD can be suppressed within a range in which the decrease in the enzyme activity is industrially acceptable. In view of productivity, step I is preferably performed at 4° C. or higher, and more preferably 4° C. or higher but 8° C. or lower. This is because the rate of the progress of the chemical reaction is significantly decreased at lower than 4° C.

The polymerizable functional group 2 is not particularly limited as long as the polymerizable functional group 2 can be introduced onto the surface of a BOD molecule. For example, the polymerizable functional group 2 may be radical-polymerizable, cationic-polymerizable, anionic-polymerizable, addition-polymerizable, or condensation-polymerizable. Specific examples of the polymerizable functional group 2 include an acryloyl group, a methacryloyl group, a vinyl group, an allyl group, a propenyl group, an acrylamide group, a vinylamide group, a vinylidene group, and a vinylene group. As an example, step I of introducing a polymerizable functional group in the case where an acryloyl group is introduced onto a BOD molecule is shown by reaction formula 1 below. In order to facilitate understanding of the state of covalent bond between an amino group present on the surface of the BOD molecule and the acryloyl group, the amino group present on the surface of the BOD molecule is specifically shown.

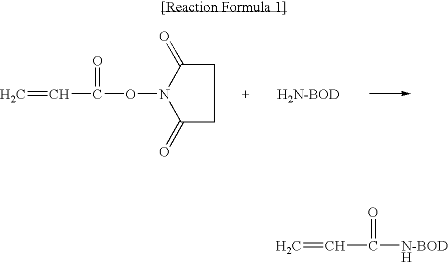

In step I of introducing a polymerizable functional group, a stabilizer of BOD may be optionally added. The stabilizer is not particularly limited, but, for example, ethylenediaminetetraacetic acid (EDTA), dimethylaminoantipyrine, or aspartic acid can be used as the stabilizer of BOD. This stabilizer is used for the purpose of stabilization of copper ions, which serve as active center ions of BOD, or inhibition of proteases.

Step II of Copolymerization

In the coating method according to this embodiment, after step I of introducing a polymerizable functional group is performed, a step of causing a copolymerization reaction between the introduced polymerizable functional group 2 and a polymerizable monomer is performed. Any method in the related art can be used for the copolymerization reaction in step II of copolymerization. For example, a polymerization initiator or a reaction accelerator may be added to BOD in which the polymerizable functional groups 2 have been introduced onto the surface of the molecule to initiate copolymerization of the polymerizable functional groups 2 on the surface of the BOD molecule, and the polymerizable monomer and a cross-linking agent may be added thereto. Thus, the copolymerization reaction can be performed.

Step II of copolymerization is performed at 17° C. or lower. This is because a decrease in the enzyme activity of BOD is suppressed. Hitherto, a copolymerization reaction has been generally performed at about 25° C. However, in this embodiment, the idea of the related art is significantly changed, and by performing the copolymerization reaction at a relatively low temperature of 17° C. or lower, the decrease in the enzyme activity of BOD can be successfully suppressed.

When step II of copolymerization is performed at 17° C. or lower, the decrease in the enzyme activity of BOD can be suppressed within a range in which the decrease in the enzyme activity is industrially acceptable. In view of productivity, step II is preferably performed at 4° C. or higher, and more preferably 4° C. or higher but 8° C. or lower. This is because the rate of the progress of the copolymerization reaction is significantly decreased at lower than 4° C.

In step II of copolymerization, the polymerizable monomer used for the copolymerization with the polymerizable functional group 2 introduced onto the surface of a BOD molecule is not particularly limited as long as the copolymerization with the polymerizable functional group can be performed. Examples of the polymerizable monomer include acrylamide, acrylic acid, methacrylic acid, 2-methacryloyloxyethyl phosphorylcholine (MPC), N-isopropylacrylamide, 2-hydroxyethyl methacrylate, N-vinylpyrrolidone, and 2-aminoethyl methacrylate.

In step II of copolymerization, for example, a polymerization initiator, a reaction accelerator, or a cross-linking agent may be optionally used. Examples of the polymerization initiator include ammonium sulfate, benzoyl peroxide (BPO), azobisisobutyronitrilc (AIBN), and tetramethylethylcnediamine (TMED). An example of the reaction accelerator is tetramethylethylenediamine (TMED). Examples of the cross-linking agent include N,N-methylenebisacrylamide (MBAAm) and ethyleneglycol dimethacrylate (EDMA).

In the method of coating BOD according to an embodiment, a BOD molecule can be coated and stabilized while suppressing a decrease in the enzyme activity of BOD. When an enzyme is coated with a polymer or the like for the purpose of stabilization using a coating method in the related art, the enzyme activity is decreased. In contrast, in the coating method according to the embodiment, a BOD molecule is coated and stabilized while suppressing the decrease in the enzyme activity of BOD. Therefore, the method according to the embodiment is advantageous in that BOD that is highly stabilized and that has a high enzyme activity can be prepared.

In the coating method according to the embodiment, since BOD molecules are individually coated, the BOD molecules can be stabilized in terms of various characteristics. For example, BOD can be stabilized by improving thermostability. In the embodiment, the improvement in thermostability can be achieved both when the heat treatment temperature is increased and the heat treatment time is increased.

Alternatively, BOD can be stabilized by improving the resistance against an organic solvent. In this case, examples of the organic solvent include, but are not particularly limited to, alcohols such as methanol, ethers, acetone, hexane, tetrahydrofuran (THF), and dioxane.

Coated BOD produced by the coating method according to the embodiment exhibits an enzyme activity, and in addition, has high stability against heat, organic solvents, or the like. Accordingly, the coated BOD can be suitably used for all applications. In particular, the coated BOD can be effectively used for, for example, an application in which heat is generated, or an application in which the BOD is used in the form of an organic solvent solution.

As a specific example of the application, by immobilizing the coated BOD at the cathode (positive electrode) side of biofuel cell, the coated BOD can be used as a catalyst that realizes an electrochemical four-electron reduction reaction of oxygen. As long as the coated BOD produced by the method according to an embodiment is immobilized in an enzyme-immobilized electrode, other structures and functions of the enzyme-immobilized electrode are not particularly limited.

Since the BOD according to an embodiment, the BOD has high stability and an decrease in the enzyme activity when the BOD is immobilized on an electrode can also be suppressed. For example, the BOD according to the embodiment has high stability against heat, the durability of the resulting enzyme-immobilized electrode can be improved.

In addition, it has been difficult to dissolve BOD in an organic solvent and to immobilize the BOD on an electrode because a decrease in the enzyme activity is a matter of concern. However, the BOD according to an embodiment has high stability against organic solvents. Accordingly, the BOD according to an embodiment is advantageous in that an enzyme immobilization method using an organic solvent can also be employed.

The enzyme-immobilized electrode according to an embodiment can be used for any fuel cell in the related art. For example, the type of fuel, the structure, and the function of the fuel cell are not limited as long as at least the enzyme-immobilized electrode according to an embodiment can be used.

The BOD used in the enzyme-immobilized electrode of a fuel cell according to an embodiment has high stability against heat, organic solvents, or the like, and thus the stability and the durability of the fuel cell itself can be improved.

### REFERENCE EXAMPLE

In Reference Example, changes in the enzyme activity and thermostability in the case where bilirubin oxidase (hereinafter referred to as “BOD”) was coated using the same method as that in the related art were examined.

Introduction of Polymerizable Functional Group

In Reference Example, as an example of a polymerizable functional group, an acryloyl group was introduced.

First, 10 mg of BOD and 1 mg of 4-dimethylaminoantipyrine used as a stabilizer were dissolved in 3.8 mL of a 100 mM boric acid solution (pH 9.3). Subsequently, 4.0 mg of N-acryloxysuccinimide was dissolved in 0.2 mL of dimethyl sulfoxide (DMSO), and the solution was then gradually added to the boric acid buffer. The resulting solution was allowed to react at 30° C. for two hours. Subsequently, the BOD in which acryloyl groups were introduced (hereinafter referred to as “acryloylated BOD”) was purified using a Sephadex G25 column.

Measurement of Acryloylation Ratio of BOD

First, 0.5 mL of a solution of acryloylated BOD purified above (prepared by dissolving in a 0.1 M phosphate buffer with a pH of 8.0) was added to a 0.1 mg/mL fluorescamine solution (prepared by dissolving in 1.5 mL of acetone), and the resulting solution was allowed to react at 25° C. for seven minutes. Subsequently, the fluorescence intensity at an excitation wavelength of 390 nm and an emission wavelength of 483 nm was measured using a fluorescence spectrophotometer. In order to determine the amount of unreacted amine residue, BOD in which no polymerizable functional group was introduced (hereinafter referred to as “free BOD”) was used as a control. The acryloylation ratio of acryloylated BOD in which acryloyl groups were introduced as described above was 55.9%.

Copolymerization

Acryloylated BOD and 4-dimethylaminoantipyrine were mixed in a vial so that the total volume of the mixture was 3.5 mL, and oxygen was removed. Next, a solution prepared by dissolving ammonium sulfate (3 mg) in 30 μL of deionized water free of oxygen, and 3 μL of N,N,N′,N′-tetramethylethylenediamine were added to the vial to initiate polymerization on the surface of acryloylated BOD.

Subsequently, a mixture prepared by mixing acrylamide and N,N′-methylenebisacrylamide in a molar ratio of 10:1 was dissolved in 0.5.mL of deionized water free of oxygen. The solution was homogeneously added to the above acryloylated BOD solution, and the resulting solution was allowed to react for 60 minutes. The reaction was further performed in the presence of nitrogen for 60 minutes. Finally, the coated BOD (hereinafter referred to as “coated BOD 1”) was purified using a Sephadex G75 column. In coated BOD 1, the molar ratio of the mixture of acrylamide and N,N′-methylenebisacrylamide, and BOD was 400:1.

The above method was further repeatedly performed using purified coated BOD 1 in a molar ratio of the mixture of acrylamide and N,N′-methylenebisacrylamide, and BOD of 400:1, thus allowing coated BOD (hereinafter referred to as “coated BOD 2”) to be prepared.

As a control, BOD on which coating was not performed and which was stored at 4° C. for the same period of time as that is necessary for the process of preparing coated BOD 2 (hereinafter referred to as “free BOD 1”) was used.

Measurement of Enzyme Activity

The Michaelis constants (K_{M}) and the reaction rate constants (k_{cat}) of coated BOD 1, coated BOD 2, and free BOD 1 prepared above were measured under the conditions shown in Table 1 below. 2,2′-Azino-bis(3-ethylbenzothiazoline-6-sulfonic acid) diammonium salt (ABTS) was used as a substrate. The amount of protein of each BOD sample was determined by bicinchoninic acid (BCA) colorimetric protein assay from a calibration curve of bovine serum albumin (BSA) used as a standard protein.

TABLE 1

| | |
|---|---|
| Coated BOD 1 | 2.05 nM |
| Coated BOD 2 | 19.7 nM |
| Free BOD 1 | 712 pM |
| ABTS | 0.05 to 10 mM |
| Solvent | 46.5 mM, pH 7.0 sodium phosphate solution |
| Measurement | 25° C., Oxygen saturation |
| temperature/condition | |
| Measurement wavelength | 730 nm |

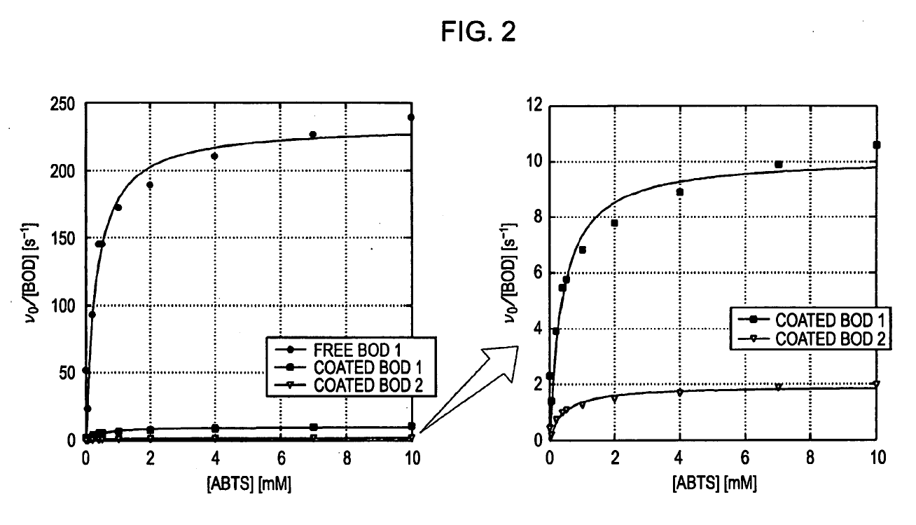

The results are shown in FIG. 2. FIG. 2 includes graphs showing the relationship between the substrate concentration and the enzyme activity of coated BOD 1, coated BOD 2, and free BOD 1, the relationship being obtained by performing fitting using the Michaelis-Menten equation. The Michaelis constants (K_{M}) and the reaction rate constants (k_{cat}) of coated BOD 1, coated BOD 2, and free BOD 1 determined from FIG. 2 are shown in Table 2.

TABLE 2

| | Coated BOD 1 | Coated BOD 2 | Free BOD 1 |
|---|---|---|---|
| K_{M,ABTS} (μM) | 383 | 405 | 307 |
| k_{cat} (s⁻¹) | 10.2 | 1.92 | 234 |
| k_{cat}/K_{M,ABTS} | 0.27 | 0.048 | 7.6 |
| (M⁻¹s⁻¹ × 10⁵) | | | |

When the Michaelis constants (K_{M}) and reaction rate constants (k_{cat}) of coated BOD 1 and coated BOD 2 were compared with those of free BOD 1, there was no significant difference in the Michaelis constants (K_{M}), but the reaction rate constant (k_{cat}) of coated BOD 1 was about 1/23 times that of free BOD 1 and the reaction rate constant (k_{cat}) of coated BOD 2 was about 1/122 times that of free BOD 1.

Thermostability Test: Dependency of Enzyme Activity on the Heat Treatment Time

In a 46.5 mM sodium phosphate solution with a pH of 7.0, 2.46 μM of coated BOD 1, 11.8 μM of coated BOD 2, or 1.07 μM of free BOD 1 was dissolved, and each of the resulting solutions was heat-treated on a hot plate at 65° C. After the heat treatment, the enzyme activities were measured under the conditions shown in Table 3.

TABLE 3

| | |
|---|---|
| Coated BOD 1 | 4.09 nM |
| Coated BOD 2 | 19.7 nM |
| Free BOD 1 | 1.78 nM |
| ABTS | 3.0 mM |
| Solvent | 46.5 mM, pH 7.0 sodium phosphate solution |
| Measurement | 25° C., Oxygen saturation |
| temperature/condition | |

Here, a thermal denaturation reaction of a protein is represented by reaction formula 2 below. If the relationship k₂>>k₁ is satisfied, the thermal denaturation reaction of the protein is represented by reaction formula 3 below, and a rate analysis formula of a first-order reaction is represented by equation 1 below. Consequently, fitting was performed using the rate analysis formula represented by equation 1.

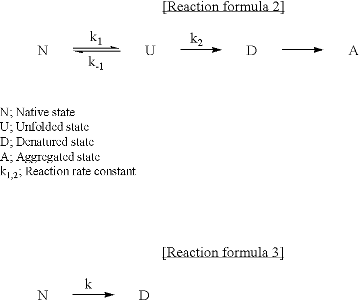

[N]=[N]₀e^{-kt} [Equation 1]

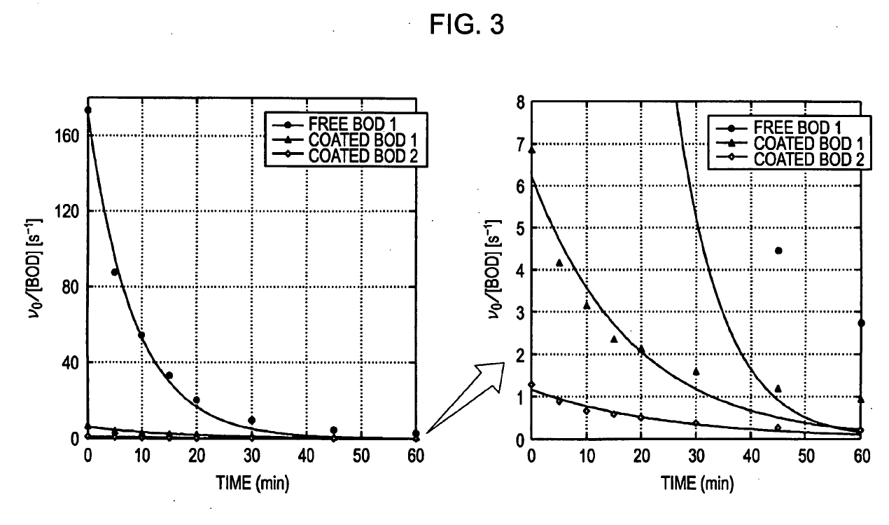

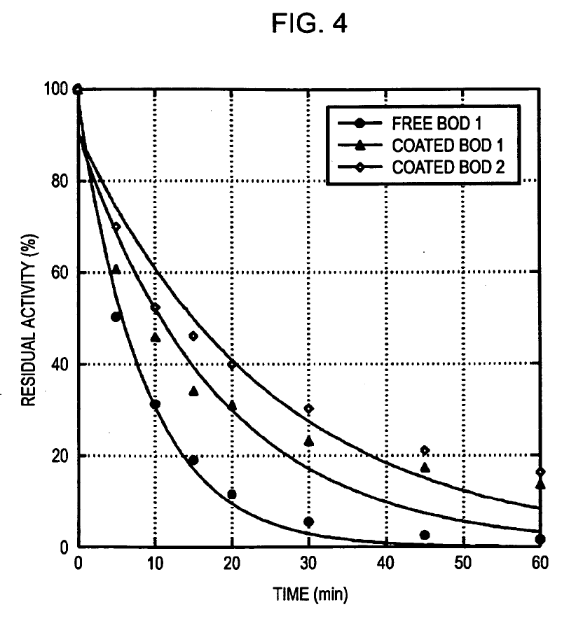

The results are shown in FIGS. 3 and 4. FIG. 3 includes graphs showing the relationship between the heat treatment time and the enzyme activity of coated BOD 1, coated BOD 2, and free BOD 1, the relationship being obtained by performing fitting using the rate analysis equation represented by equation 1. FIG. 4 is a graph showing the relationship between the heat treatment time and the residual activity of coated BOD 1, coated BOD 2, and free BOD 1, the relationship being obtained by performing fitting using the rate analysis equation represented by equation 1.

When the residual activities of coated BOD 1, coated BOD 2, and free BOD 1 at a heat treatment time of 60 minutes are compared with each other, the residual activity of free BOD 1 was about 0% whereas the residual activities of coated BOD 1 and coated BOD 2 were about 15%.

In Reference Example, it was found that by coating BOD using the same method as that used in the related art, thermostability was improved but the enzyme activity was significantly decreased.

### Example 1

In Example 1, changes in the enzyme activity and thermostability of BOD in the case where the step of introducing a polymerizable functional group and the step of copolymerization in the coating method of Reference Example were performed at 4° C.

Introduction of Polymerizable Functional Group

In this Example, as an example of a polymerizable functional group, an acryloyl group was introduced.

First, 40 mg of BOD and 1 mg of ethylenediaminetetraacetic acid (EDTA) used as a stabilizer were dissolved in 3.8 mL of a 100 mM boric acid solution (pH 9.3). Subsequently, 6.0 mg of N-acryloxysuccinimide was dissolved in 0.2 mL of DMSO, and the solution was then gradually added to the boric acid buffer. The resulting solution was allowed to react at 4° C. for 10 hours. Subsequently, the resulting acryloylated BOD in which acryloyl groups were introduced was purified using a Sephadex G25 column at 4° C.

Measurement of Acryloylation Ratio of BOD

The acryloylation ratio of BOD was measured by the same method as that used in Reference Example. The acryloylation ratio of acryloylated BOD in which acryloyl groups were introduced as described above was 60.6%.

Copolymerization

Acryloylated BOD and EDTA were mixed in a vial at 4° C. so that the total volume of the mixture was 3.5 mL, and oxygen was removed. Next, a solution prepared by dissolving ammonium sulfate (3 mg) in 30 μL of deionized water free of oxygen, and 3 μL of N,N,N′,N′-tetramethylethylenediamine were added to the vial to initiate polymerization on the surface of acryloylated BOD.

Subsequently, at 4° C., a mixture prepared by mixing acrylamide and N,N′-methylenebisacrylamide in a molar ratio of 10:1 was dissolved in 0.5 mL of deionized water free of oxygen. The solution was homogeneously added to the above acryloylated BOD solution, and the resulting solution was allowed to react for 60 minutes. The reaction was further performed in the presence of nitrogen at 4° C. for 60 minutes. Finally, the coated BOD (hereinafter referred to as “coated BOD 3”) was purified using a Sephadex G75 column. In coated BOD 3, the molar ratio of the mixture of acrylamide and N,N′-methylenebisacrylamide, and BOD was 400:1.

The above method was further repeatedly performed using purified coated BOD 3 in a molar ratio of the mixture of acrylamide and N,N′-methylenebisacrylamide, and BOD of 400:1, thus allowing coated BOD (hereinafter referred to as “coated BOD 4”) to be prepared.

As controls, BOD on which coating was not performed and which was stored at 25° C. for the same period of time as that is necessary for the process of preparing coated BOD 4 (hereinafter referred to as “free BOD 2”), and BOD on which coating was not performed and which was stored at 4° C. for the same period of time as that is necessary for the process of preparing coated BOD 4 (hereinafter referred to as “free BOD 3”) were used.

Measurement of Enzyme Activity

The Michaelis constants (K_{M}) and the reaction rate constants (k_{cat}) of coated BOD 3, coated BOD 4, free BOD 2, and free BOD 3 prepared above were measured under the conditions shown in Table 4. 2,2′-Azino-bis(3-ethylbenzothiazoline-6-sulfonic acid) diammonium salt (ABTS) was used as a substrate. The amount of protein of each BOD sample was determined by BCA colorimetric protein assay from a calibration curve of BSA used as a standard protein.

TABLE 4

| | |
|---|---|
| Coated BOD 3 | 1.09 nM |
| Coated BOD 4 | 3.30 nM |
| Free BOD 2 | 0.603 nM |
| Free BOD 3 | 0.629 nM |
| ABTS | 0.05 to 7 mM |
| Solvent | 46.5 mM, pH 7.0 sodium phosphate solution |
| Measurement | 25° C., Oxygen saturation |
| temperature/condition | |
| Measurement wavelength | 730 nm |

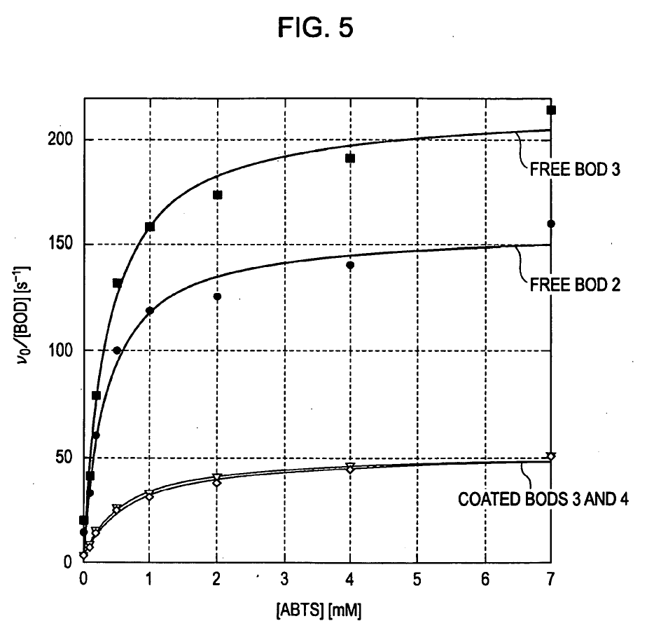

The results are shown in FIG. 5. FIG. 5 is a graph showing the relationship between the substrate concentration and the enzyme activity of coated BOD 3, coated BOD 4, free BOD 2, and free BOD 3, the relationship being obtained by performing fitting using the Michaelis-Menten equation. The Michaelis constants (K_{M}) and the reaction rate constants (k_{cat}) of coated BOD 3, coated BOD 4, free BOD 2, and free BOD 3 determined from FIG. 5 are shown in Table 5.

TABLE 5

| | Coated | | | |
|---|---|---|---|---|
| | BOD 3 | Coated BOD 4 | Free BOD 2 | Free BOD 3 |
| K_{M,ABTS} (μM) | 570 | 670 | 337 | 358 |
| k_{cat} (s⁻¹) | 52.5 | 52.7 | 158 | 216 |
| k_{cat}/K_{M,ABTS} | 0.92 | 0.79 | 4.7 | 6.0 |
| (M⁻¹s⁻¹ × 10⁵) | | | | |

When the Michaelis constants (K_{M}) and reaction rate constants (k_{cat}) of coated BOD 3 and coated BOD 4 were compared with those of free BOD 2 and free BOD 3, the Michaelis constants (K_{M}) of coated BOD 3 and coated BOD 4 were about twice those of free BOD 2 and free BOD 3. In this case, the reaction rate constants (k_{cat}) of coated BOD 3 and coated BOD 4 were about ⅓ times that of free BOD 2 and about ¼ times that of free BOD 3.

Thermostability Test: Dependency of Enzyme Activity on the Heat Treatment Time

In a 46.5 mM sodium phosphate solution with a pH of 7.0, 1.14 μM of coated BOD 3, 1.98 μM of coated BOD 4, 0.362 μM of free BOD 2, or 0.377 μM of free BOD 3 was dissolved, and each of the resulting solutions was heat-treated on a hot plate at 65° C. After the heat treatment, the enzyme activities were measured under the conditions shown in Table 6.

TABLE 6

| | |
|---|---|
| Coated BOD 3 | 1.90 nM |
| Coated BOD 4 | 3.30 nM |
| Free BOD 2 | 0.603 nM |
| Free BOD 3 | 0.629 nM |
| ABTS | 5.0 mM |
| Solvent | 46.5 mM, pH 7.0 sodium phosphate solution |
| Measurement | 25° C., Oxygen saturation |
| temperature/condition | |

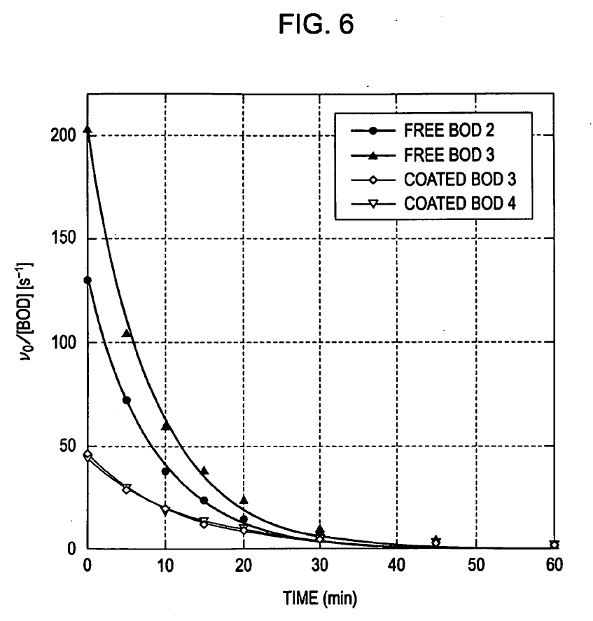

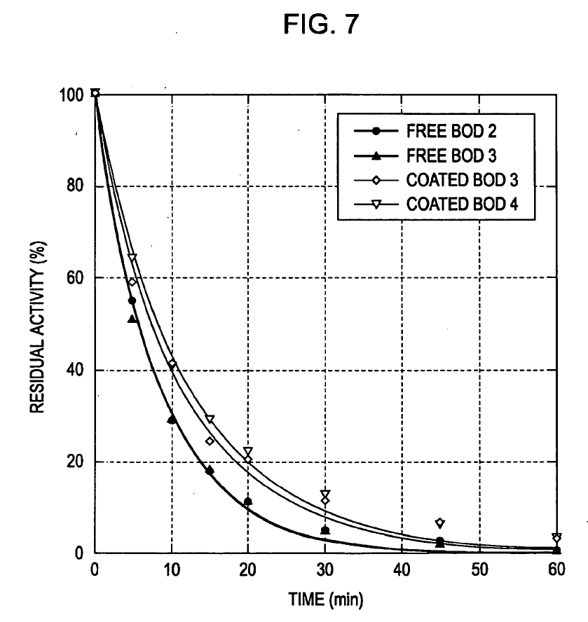

The results are shown in FIGS. 6 and 7. FIG. 6 is a graph showing the relationship between the heat treatment time and the enzyme activity of coated BOD 3, coated BOD 4, free BOD 2, and free BOD 3, the relationship being obtained by performing fitting using the rate analysis equation represented by equation I above. FIG. 7 is a graph showing the relationship between the heat treatment time and the residual activity of coated BOD 3, coated BOD 4, free BOD 2 and free BOD 3, the relationship being obtained by performing fitting using the rate analysis equation represented by equation 1.

As shown in FIG. 7, the curves of the residual activity of coated BOD 3 and coated BOD 4 are shifted from the curves of the residual activity of free BOD 2 and free BOD 3 with the increase in the heat treatment time in the direction in which time increases along the horizontal axis of the graph. That is, these results showed that thermostability for the heat treatment time was improved in coated BOD 3 and coated BOD 4.

Thermostability Test: Dependency of Enzyme Activity on the Heat Treatment Temperature

Coated BOD 3, coated BOD 4, free BOD 2, or free BOD 3 was dissolved in a 46.5 mM sodium phosphate solution with a pH of 7.0, and the concentration of each of the solutions was adjusted to about 1.9 μM. Each of the adjusted solutions was heat-treated on a hot plate at 25° C., 40° C., 50° C., 55° C., 60° C., 65° C., 70° C., 75° C., and 80° C. for 10 minutes. After the heat treatment, the enzyme activities were measured under the conditions shown in Table 6.

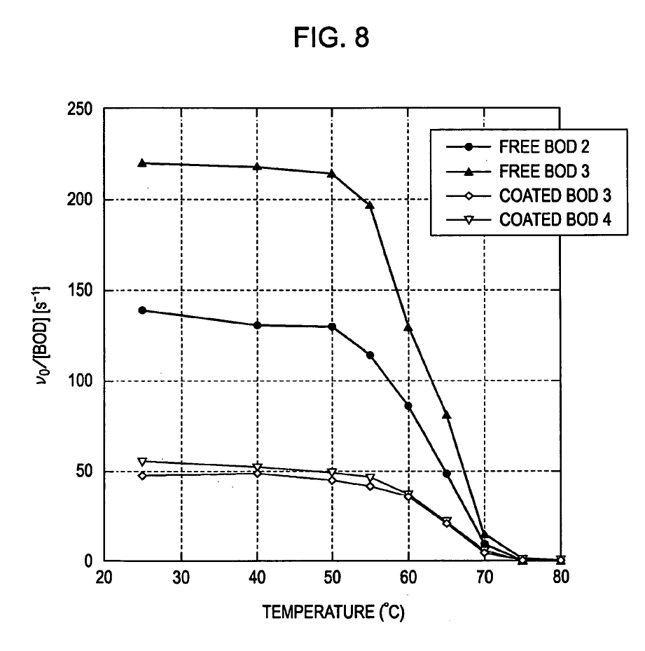

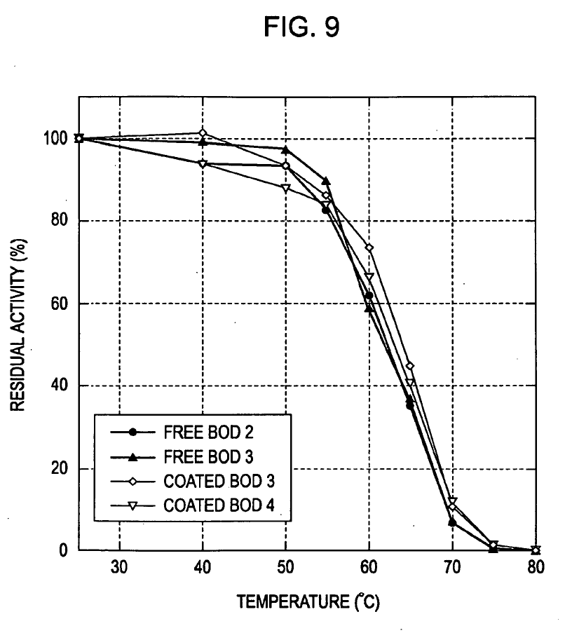

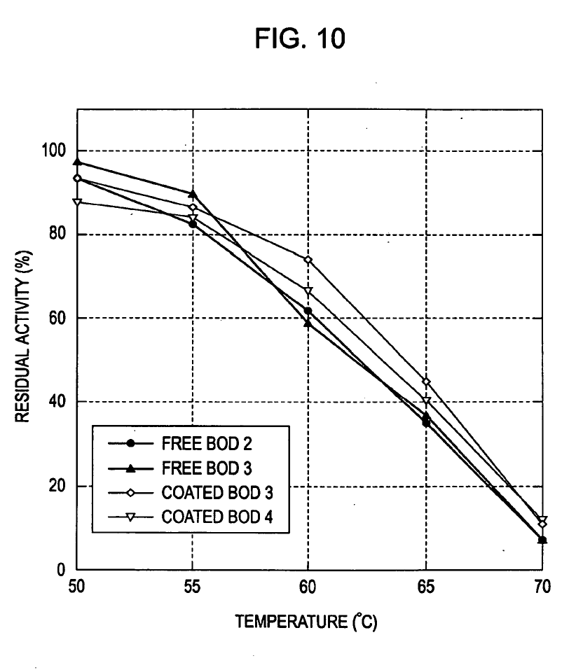

The results are shown in FIGS. 8, 9, and 10. FIG. 8 is a graph showing the relationship between the heat treatment temperature and the enzyme activity of coated BOD 3, coated BOD 4, free BOD 2, and free BOD 3. FIG. 9 is a graph showing the relationship between the heat treatment temperature and the residual activity of coated BOD 3, coated BOD 4, free BOD 2, and free BOD 3. FIG. 10 is an enlarged graph showing the range from 50° C. to 70° C. in FIG. 9.

As shown in FIG. 9, the curves of the residual activity of coated BOD 3 and coated BOD 4 are shifted from the curves of the residual activity of free BOD 2 and free BOD 3 with the increase in the heat treatment temperature in the direction in which temperature increases along the horizontal axis of the graph. That is, these results showed that thermostability for the heat treatment temperature was improved in coated BOD 3 and coated BOD 4.

In addition, a heat treatment temperature at which the residual activity is 50% was defined as a denaturation middle point (Tm), and the denaturation middle point (Tm) of each of the BOD samples was determined from FIG. 10. The results are shown in Table 7.

TABLE 7

| | Tm (° C.) |
|---|---|
| Coated BOD 3 | 64.5 |
| Coated BOD 4 | 63.5 |
| Free BOD 2 | 62.0 |
| Free BOD 3 | 62.0 |

As shown in Table 7, the denaturation middle points (Tm) of coated BOD 3 and coated BOD 4 were higher than those of free BOD 2 and free BOD 3. That is, these results showed that thenmostability for the heat treatment temperature was improved in coated BOD 3 and coated BOD 4.

Improvement in thermostability was observed in the case where BOD was coated using the method (in the related art) of Reference Example and in the case where BOD was coated using the method of Example 1. However, in the case where BOD was coated using the method (in the related art) of Reference Example, the enzyme activity was significantly decreased. In contrast, it was found that, in case where BOD was coated using the method of Example 1, a decrease in the enzyme activity could be suppressed.

Accordingly, in a method of coating BOD, by performing the step of introducing a polymerizable functional group and the step of copolymerization at a low temperature, thermostability can be improved while suppressing a decrease in the enzyme activity of BOD.

### Example 2

In Example 2, the upper temperature limit of the method of coating BOD according to an embodiment was determined.

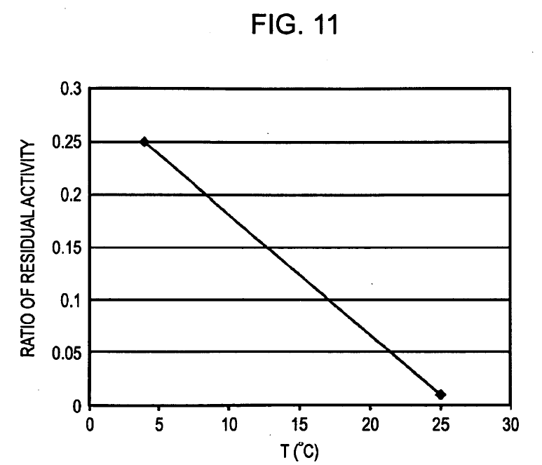

As described in Reference Example, when BOD was coated at 25° C., the enzyme activity of BOD was significantly decreased to about 1/100. In contrast, as described in Example 1, when BOD was coated at 4° C., the decrease in the enzyme activity could be suppressed to about ¼. Consequently, it is assumed that the temperature T (° C.) during the process of BOD coating and the ratio of residual activity has the relationship shown in FIG. 11.

When the ratio of decrease in BOD activity that is industrially acceptable was assumed to be 1/10, the upper temperature T (° C.) limit of the BOD coating process was determined to be about 17° C. Furthermore, as a more preferable temperature, when the ratio of decrease in BOD activity was assumed to be ⅕, the upper temperature T (° C.) limit of the BOD coating process was determined to be about 8° C.

### Example 3

In Example 3, organic solvent resistance of coated BOD samples produced by the coating method according to an embodiment was examined.

In a 46.5 mM sodium phosphate solution with a pH of 7.0, the sodium phosphate solution containing 15% of methanol, 1.6 μM of coated BOD 3, coated BOD 4, or free BOD 2, which was prepared in Example 1, was dissolved, and each of the resulting solutions was heat-treated at 60° C. for 10 minutes. After the heat treatment, the enzyme activities were measured under the conditions shown in Table 8 below.

TABLE 8

| | |
|---|---|
| Coated BOD 3 | 1.35 nM |
| Coated BOD 4 | 2.80 nM |
| Free BOD 2 | 0.512 nM |
| ABTS | 5.0 mM |
| Solvent | 46.5 mM, pH 7.0 sodium phosphate solution |
| Measurement | 25° C., Oxygen saturation |
| temperature/condition | |

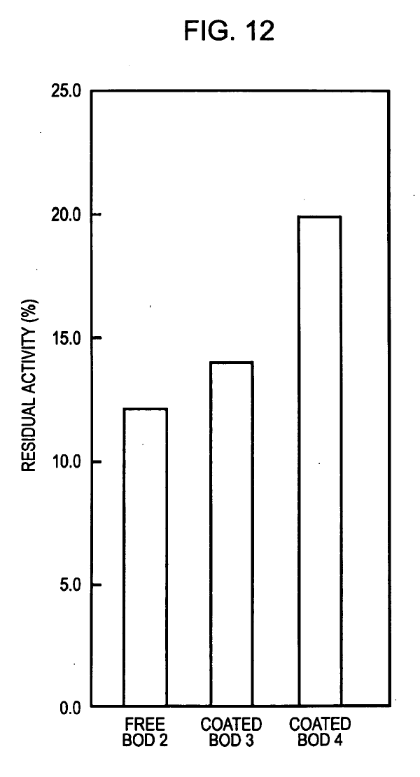

The results are shown in FIG. 12. As shown in FIG. 12, the residual activity of free BOD 2 after the organic solvent treatment was about 12%, whereas the residual activity of coated BOD 3 was about 14% and that of coated BOD 4 was about 20%. These results showed that organic solvent resistance of coated BOD 3 was increased by about 2% relative to that of free BOD 2, and organic solvent resistance of coated BOD 4 was increased by about 8% relative to that of free BOD 2.

According to the results in Example 3, by coating BOD using the coating method according to an embodiment, organic solvent resistance of BOD can be improved.

It should be understood by those skilled in the art that various modifications, combinations, sub-combinations and alterations may occur depending on design requirements and other factors insofar as they are within the scope of the appended claims or the equivalents thereof.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of coating bilirubin oxidase (BOD) comprising:
introducing a polymerizable functional group onto the surface of a bilirubin oxidase (BOD) molecule; and
copolymerizing the polymerizable functional group with a polymerizable monomer,
wherein introducing the polymerizable functional group and copolymerizing the polymerizable functional group with the polymerizable monomer are performed at 17° C. or lower.

2. The method according to claim 1,
wherein introducing the polymerizable functional group and copolymerizing the polymerizable functional group with the polymerizable monomer are performed at 4° C. or higher but 17° C. or lower.

3. The method according to claim 1, wherein the polymerizable functional group is an acryloyl group.

4. The method according to claim 1, wherein the polymerizable monomer is acrylamide.

5. A method of stabilizing bilirubin oxidase (BOD) comprising:
introducing a polymerizable functional group onto the surface of a bilirubin oxidase (BOD) molecule; and
copolymerizing the polymerizable functional group with a polymerizable monomer,
wherein introducing the polymerizable functional group and copolymerizing the polymerizable functional group with the polymerizable monomer are performed at 17° C. or lower.

6. The method according to claim 5, wherein a thermostability of bilirubin oxidase (BOD) is improved.

7. The method according to claim 5, wherein a resistance against an organic solvent of bilirubin oxidase (BOD) is improved.

8. The method according to claim 7,
wherein the organic solvent is methanol.

9. A bilirubin oxidase (BOD) coated by:
introducing a polymerizable functional group onto the surface of a bilirubin oxidase (BOD) molecule; and
copolymerizing the polymerizable functional group with a polymerizable monomer,
wherein introducing the polymerizable functional group and copolymerizing the polymerizable functional group with the polymerizable monomer are performed at 17° C. or lower.

10. An enzyme-immobilized electrode comprising:
a bilirubin oxidase (BOD) coated by:
introducing a polymerizable functional group onto the surface of a bilirubin oxidase (BOD) molecule; and
copolymerizing the polymerizable functional group with a polymerizable monomer,
wherein introducing the polymerizable functional group and copolymerizing the polymerizable functional group with the polymerizable monomer are performed at 17° C. or lower; and
wherein the bilirubin oxidase (BOD) is immobilized.

11. A fuel cell comprising:
an enzyme-immobilized electrode including a bilirubin oxidase (BOD) coated by
introducing a polymerizable functional group onto the surface of a bilirubin oxidase (BOD) molecule, and
copolymerizing the polymerizable functional group with a polymerizable monomer,
wherein introducing the polymerizable functional group and copolymerizing the polymerizable functional group with the polymerizable monomer are performed at 17° C. or lower.
